(19) United States
(12) Patent Application Publication
(10) Pub. No.: US 2014/0301984 A1
(43) Pub. Date: Oct. 9, 2014

(54) METHOD FOR THE USE OF NITRATE REDUCING BACTERIA AND PHAGES FOR MITIGATING BIOGENIC SULFIDE PRODUCTION

(71) Applicant: Multi-Chem Group, LLC, San Angelo, TX (US)

(72) Inventors: Edward Corrin, Houston, TX (US); Michael Harless, Humble, TX (US)

(73) Assignee: MULTI-CHEM GROUP, LLC, San Angelo, TX (US)

(21) Appl. No.: 13/857,698

(22) Filed: Apr. 5, 2013

(51) Int. Cl. A61K 35/76; A61K 33/00

(52) U.S. Cl. CPC A61K 35/76; A61K 33/00
USPC 424/93.6

(57) ABSTRACT

A method of controlling a deleterious bacteria in a fluid including injecting a nitrite or nitrate into the fluid, identifying a phage capable of infecting the deleterious bacteria, and injecting the phage into the fluid.

### BACKGROUND OF THE DISCLOSURE

1. Field of the Disclosure

The disclosure relates generally to the field of souring and microbiologically influenced corrosion in oil and gas production and completion fluids, as well as other industrial waters. More specifically the disclosure relates to methods for controlling souring and microbiologically influenced corrosion by deleterious microbes.

2. Background Art

Oil and gas production and completion fluids, as well as other industrial fluids, suffer corrosion, pipe necking (partial blockage) and scale buildup in pipes and pipelines. Sources of these problems include microbially influenced corrosion (MIC) corrosion, solids produced by metabolite byproducts, and bio-film blockages. Microbes may also negatively affect oil and natural gas recovery through bacterial fouling of the water needed to hydrofracture (“frac”) reservoir rock or to “water-flood,” to increase production of oil and gas. One particular type of microbe, sulfate reducing bacteria (SRB) can contaminate or “sour” the reservoir by producing hydrogen sulfide (H₂S). SRBs may produce toxic and flammable H₂S, which may shorten the lifetime of piping and tankage, and introduce additional safety risks from drill rig to refinery. This H₂S may react with soluble iron to produce iron sulfide. Acid producing bacteria (APB) produce acids, including a variety of organic acids, which lead to additional corrosion. SRBs and APBs may have the same effects in other oil and gas completion fluids, as well as other industrial fluids.

### SUMMARY

In one embodiment of the present disclosure, a method of controlling a deleterious bacteria in a fluid is disclosed. The method includes injecting a nitrite or nitrate into the fluid, identifying a phage capable of infecting the deleterious bacteria, and injecting the phage into the fluid.

### DETAILED DESCRIPTION

The following disclosure provides many different embodiments, or examples, for implementing different features of various embodiments. Specific examples of components and arrangements are described below to simplify the present disclosure. These are, of course, merely examples and are not intended to be limiting.

Various terms as used herein are shown below. To the extent a term used in a claim is not defined below, it should be given the broadest definition skilled persons in the pertinent art have given that term as reflected in printed publications and issued patents at the time of filing. Further, unless otherwise specified, all compounds described herein may be substituted or unsubstituted and the listing of compounds includes derivatives thereof.

Further, various ranges and/or numerical limitations may be expressly stated below. It should be recognized that unless stated otherwise, it is intended that endpoints are to be interchangeable. Further, any ranges include iterative ranges of like magnitude falling within the expressly stated ranges or limitations.

In certain embodiments of the present disclosure, nitrates or nitrites may be injected into the oil and gas or other industrial fluids in conjunction and bacteriophages adapted to lyse SRB as a control mechanism for SRB. In other embodiments, nitrate-reducing bacteria or nitrate reducing sulfide oxidizing bacteria (NRSOB) (generically, “NRB”) and the bacteriophages adapted to lyse SRB may be injected into the oil and gas or other industrial fluids, and may be used in conjunction with the nitrates or nitrites The nitrates and nitrites may be organic or inorganic. Molybdates also may also be used in these systems as a control mechanism for SRB. Further, in certain embodiments, biocides may be used.

SRB and NRB typically compete for the same non-polymer carbon source (such as acetates) present in certain oilfield and industrial water systems needed for growth of bacteria. By increasing the growth rate of the NRB in comparison to the SRB, the NRB may out compete the SRB in consumption of the available non-polymer carbon source, depriving the SRB of its ability to grow and create the undesirable sulfides and reduce corrosion rates. Further, by inhibiting the growth rate of the SRB, the NRB may predominate, again out competing the SRB for the available non-polymer carbon in the certain oilfield and industrial water systems.

Organic and inorganic nitrates and nitrites serve to stimulate the growth of the NRB present in the certain oilfield and industrial water systems, thus outcompeting SRB present in the formation. Organic and inorganic nitrates or inorganic nitrites may be used injected into the certain oilfield and industrial water systems. Inorganic nitrates and inorganic nitrites available for use in the present disclosure include, for instance, potassium nitrate, potassium nitrite, sodium nitrate, sodium nitrite, ammonium nitrate, and mixtures thereof. These organic and inorganic nitrates and inorganic nitrites are commonly available, but are non-limiting and any appropriate nitrate or nitrite may be used.

The amount of organic or inorganic nitrate or nitrite used is dependent upon a number of factors, including the amount of sulfate and/or organic acids present in the oilfield and industrial water systems, and the expected amount of NRB needed to counteract the SRB. In certain embodiments of the present disclosure, the concentration of organic or inorganic nitrate or nitrite in the oilfield or industrial water systems may be less than 2000 ppm by weight of the water solution, alternatively 500 to 1600 ppm by weight or alternatively between about 900 and 1100 ppm by weight when applied using a batch application method. When applied through continuous operation, the concentration of the organic or inorganic nitrate or nitrite may be less than 500 ppm by weight, alternatively between 10 and 500 ppm, or alternatively between 10 and 100 ppm.

In certain circumstances, such as when the indigenous amount of NRB is inadequate or wholly absent in oilfield and industrial water systems, it may be necessary to supplement the indigenous NRB with suitable additional NRB. Thus, in certain embodiments of the present disclosure, NRB are added to the certain oilfield and industrial water systems.

Those of ordinary skill in the art with the benefit of this disclosure will recognize acceptable examples of NRB appropriate for use in this disclosure. NRB include any type of microorganism capable of performing anaerobic nitrate reduction, such as heterotrophic nitrate-reducing bacteria, and nitrate-reducing sulfide-oxidizing bacteria. This may include, but is not limited to, Campylobacter sp. Nitrobacter sp., Thiobacillus sp., Nitrosomonas sp., Thiomicrospira sp., Sulfurospirillum sp., Thauera sp., Paracoccus sp., Pseudomonas sp., Rhodobacter sp., or Specific examples include, but are not limited to, Nitrobacter vulgaris, Nitrosomonas europea, Pseudomonas stutzeri, Pseudomonas aeruginosa, Paracoccus denitrificans, Sulfurospirillum deleyianum, and Rhodobacter sphaeroides.

In certain embodiments of the present disclosure, the NRB is specifically selected for the target oil and gas fluid, subterranean formation or other industrial fluid, i.e., the selection process for the NRB includes identification of strains that proliferate and metabaolize under the measured conditions of the particular system for which the NRB will be applied. These selection criteria include, but are not limited to, system temperatures, pressures, total dissolved solids concentration, anion and cation concentrations, dissolved gas concentrations, available organic carbon electron donors, and pH. The NRB that are optimized to metabolize under the system conditions may be selected from a library of existing NRB strains or may be cultured from the system to be treated or a similar system and reinjected.

The amount of NRB injected into the subterranean formation or the oil and gas or other industrial fluid may depend upon a number of factors including the amount of SRB expected, as well as any biocide that may be present. When injected into subterranean formation, the permeability and porosity of the subterranean formation may be considered as well. In certain embodiments of the present disclosure, the amount of NRB injected into the fluid is between 1 and 10⁸ bacteria count/ml of the fluid, or alternatively between 10¹ and 10⁴ bacteria count/ml of the fluid.

In addition to stimulating the NRB to out compete the SRB, it may be desirable to introduce additional SRB inhibitors in certain embodiments of the present disclosure together with the inorganic nitrates. Examples of SRB inhibitors suitable for the present disclosure are 9,10-anthraquinone, molybdates and molybdate salts, such as sodium molybdate and lithium molybdate, although any SRB inhibitor may be used in concentrations where the molybdates do not unduly affect the ability of the NRB to otherwise out compete the SRB. In certain embodiments of the present disclosure, molybdate is added to the fluid in the range of 5 to about 100 ppm by weight of fluid.

In certain embodiments of the present disclosure, a synergistic combination of bacteriophages (phages) may be used in conjunction with the NRB in oil and gas subterranean formations, production and completion fluids and other industrial fluids, to control MIC and/or souring from bacteria.

Certain phages that are capable of lysing bacteria are natural viruses that infect, reproduce within, and kill bacteria. Phage infection of bacteria may be initiated when the tail proteins of the phage recognize and adsorb to specific cell surface features of the target bacterial host. This recognition triggers the injection of the phage DNA into the bacterial cytoplasm. The genes in that phage DNA result in the synthesis and assembly of approximately 20 to 100 progeny phage particles over the course of minutes to several hours. After as little as 15 to 60 minutes, the cell is disrupted (“lysed”) as a result of phage-encoded lytic enzymes, liberating of progeny phage that can adsorb to new bacterial hosts and repeat the process.

Phages for lysing bacteria have not been known to infect plants or animals and are therefore safe to produce, store, handle and apply. Phages reproduce along with the microorganisms that they infect, and therefore may spread down-well to other bacteria of the same species that otherwise would not be affected.

In certain circumstances, deleterious bacteria may develop resistance to phages based on previous exposure or through natural selection. The resistance of the bacteria lessens the effectiveness of the phage, thereby increasing the rate and risk of MIC and souring. Initial treatment with the infective phage panel may be followed up by monitoring of the contained system to reveal the effects on the selected bacterial subpopulation. Over longer periods of time it may be necessary to alter the phages to address bacteria that have developed resistance mechanisms to the infective phages. Additionally, new bacterial species may begin to thrive in the absence of the initial selected bacterial subpopulation. Thus, the need may arise to alter the infective phage panel over time. The effectiveness of the infective phage panel may be monitored by evaluating changes in phage and bacterial host populations within the system. In such cases, it may be necessary to search for different phages to which the bacteria have not yet developed a resistance. Such a process can be time consuming and ultimately difficult after the deleterious bacteria have developed resistance to a number of different phages. However, the phage in conjunction with the NRB has a synergistic effect, thereby in certain embodiments, resulting in continuing control of the SRB over substantial periods of time or indefinitely.

In certain embodiments of the present disclosure an infective phage panel against a selected bacterial subpopulation within the contained system and delivering to either a section of the system infected with the selected bacterial subpopulation or simulating the system in laboratory conditions a series of potentially infective phages or phage panels to reduce the selected bacterial subpopulation. An effective panel is one that is considered as effective at controlling the deleterious bacteria treatment. For instance, a phage or phage panel may be considered effective if it results in a bacterial concentration drop of 4 orders of magnitude, for example, from 10⁷ to 10⁸ cfu/mL down to 10³ to 10⁴ cfu/mL. Success in reducing a bacterial population may also be measured by reduction or abatement of pipe corrosion or pipe blockage without quantifying any remaining bacterial population. The process of identifying and developing the phage or phage panel is described further below.

Once the particular deleterious bacteria is identified, a panel of phages effective against the selected subpopulation of deleterious bacteria may be identified and manufactured. Phages exhibiting bacteriolytic activity against the bacterial subpopulation are most useful. However, phages are abundant and diverse—each phage type may only attack a specific bacterial hosts and may be harmless to non-host bacteria.

Phage panels may include pre-isolated phages (i.e., phages previously known to be effective against specific types of bacteria) as well as the isolation of phages from samples taken at industrial and environmental sites. Thus, in one embodiment of the present disclosure, the step of producing the infective phage panel may include screening and isolating naturally occurring phages active against the selected bacterial population.

As the predators of particular bacteria, populations of phages are most often abundant near the bacteria upon which they prey. Therefore, when isolating particular phages effective against deleterious bacteria in a particular system, identification of an environmental site where that bacterial type is abundant often the first step.

A sample for testing a particular phage or phage panel may be a marine or freshwater sediment from an environment favorable for the growth of the host bacteria. Physiochemical properties of the sediments other surrounding of the environment to be treated may also be considered. While exact parameters will vary from host to host and environment to environment, variables to consider include salinity, temperature, pH, nitrogen or eutrophication, oxygen, and specific organic compounds.

As an alternative to identifying samples based on physiochemical properties, molecular tools can be used to identify sediments possessing wild populations of bacteria similar to the target bacteria. These methods typically require purification of DNA from the environmental sample followed by the detection of marker DNA sequences.

Phages may be isolated by a number of methods including enrichment methods or any technique involving the concentration of phages from environmental or industrial samples followed by screening the concentrate for activity against specific host targets. Given the high genetic diversity of phages, naturally occurring phages will include those with novel genomic sequence as well as those with some percent of similarity to phages known to infect other bacterial clades.

Phages can be optimized for effectiveness. Optimization of phages is accomplished by selection for naturally occurring variants, by mutagenesis and selection for desired traits, or by genetic engineering. Traits that might be optimized or altered include, but not limited to, traits involved in host range determination, growth characteristics, improving phage production, or improving traits important for the phage delivery processes. Thus, in another aspect, the step of producing the infective phage panel includes creating engineered phages against the selected bacterial population. This will include phages created for having a broad host range. This may be the product of directed genetic engineering, for example.

Small amounts of the phage or phage panel are typically ineffective in a treatment fluid for controlling deleterious bacteria. As a result, phage or phage panels may be mass produced for treatment of a large system, such as surface equipment or subterranean formations. Phage may be produced using a liquid lysate method., as described in Meese, E. et al., (1990) Nucleic Acids Res., volume 18:1923, which is incorporated herein by reference. For instance, in one production method, an exponentially (=OD600˜0.3) growing stock of the target host is produced in the volume of liquid corresponding to the desired final lysate volume. This may be accomplished by inoculating the media from a stationary stage liquid culture to a very low (OD600˜0.01) and monitoring growth specrophotometrically until the desired OD is reached. The culture may be inoculated with virus to a moi (multiplicity of infection=ratio of virus particles to individual host cells) of 0.1 to 0.001. The culture may then be incubated until lysis is observed. The lysate may then be separated into phage and both bacterial cell debris and the components of the culture media such as through vacuum filtration. Tangential flow filtration will be used to replace components of the media with, for example, a 10 mM buffer, such as, but not limited to, a phosphate buffer, and, if necessary, to concentrate the virus. The final product is an aqueous solution containing the virus particles in a weak buffer with minimal bacterial cellular debris.

After isolating the phage and selecting NRB, the system or formation may be treated to control the deleterious bacteria. Traditional methods for introducing the NRB and phage may be used. The phage and NRB may be mixed together and then introduced into the system or formation, or the phage and NRB may be introduced separately. For instance, in subterranean formations, the phage and NRB or phage/NRB mixture may be introduced as part of fracing or flooding operations. In surface systems, such as piping systems or other water systems, the phage and NRB or phage/NRB mixture may be injected into the system in effective amounts. The NRB/phage or NRB and phage may be injected into the fluid or subterranean formation in conjunction with or separately from the nitrate source. The injection may be continuous, batch, pulse or slug.

The combination of the phage with the NRB has a synergistic effect. As discussed above, bacteria may become resistant to a phage or phage panel over time. However, the combination of the phage with the NRB may significantly delay this effect and prolong the viability of the given phage or phage panel.

In certain embodiments, biocides may be used in the oil and gas and other industrial waters in conjunction with the phage and NRB. Prior to the application of the NRB, the system may be pre-treated with a biocide to reduce the existing bacterial populations, including the SRB, to allow the NRB to competitively exclude the SRB population in the fluid or subterranean formation. The NRB may be applied after the biocide concentration has been reduced to where the biocide no longer interferes or minimally interferes with the growth of the NRB. With short acting biocides, such as bleach, chlorine dioxide, DBNPA, or peracetic acid, the injection of the NRB may be after the biocide has decomposed or deteriorated. With longer acting biocides, such as a quaternary amine compound, THPS, dimethyloxazolidine, or aldehyde-based biocides, including, but not limited to gluteraldhye, it may be necessary to neutralize the biocide prior to introducing the NRB. In certain embodiments, the biocide may be compatible with the NRB and it may be possible to inject the NRB while the biocide is active. Further, in certain embodiments, the biocide is selected such that it is compatible with the bacteriophage as well.

As will be recognized by one of ordinary skill in the art with the benefit of this disclosure, many types of deleterious bacteria groups may be controlled using the disclosed method. One group of bacteria commonly associated with MIC in petroleum pipelines are SRBs. SRBs reduce sulfates to sulfides, releasing sulfuric acid and hydrogen sulfide as byproducts that react with iron that may form a black precipitate of iron sulfide. Hydrogen sulfide gas is toxic and flammable and may cause souring of the petroleum product, resulting in reduced quality and increased handling cost. The term “SRB” is a phenotypic classification and several distinct lineages of bacteria are included under this umbrella term. Bacterial subpopulations involved in the microbial influenced biocorrosion process or the oilfield souring process include those that form the corrosive products and intermediate products of sulfate reduction, including, but not limited to, hydrogen sulfide. Such populations include those forming the taxonomically varied group known as the sulfate-reducing bacteria (SRB). Bacteria selected for virus treatment include members of the SRB including, including without limitation, are members of the delta subgroup of the Proteobacteria, including Desulfobacterales, Desulfovibrionales, and Syntrophobacterales. SRBs may develop complex sessile assemblages along with other species, in biofilms attached to the inner wall of the pipeline. Biofilm forming bacteria cause pipeline corrosion, production slowdown, product quality loss (souring), potential environmental hazards, and leaks.

Bacteria selected for phage treatment also includes those that produce acidic metabolites. This specifically includes sulfur-oxidizing bacteria capable of generating sulfuric acid. These bacteria include, without limitation, sulfur bacteria such as Thiobacilli, including T. thiooxidans and T. denitrificans. Bacterial populations and isolates selected for phage treatment further includes corrosion associated iron-oxidizing bacteria. Also included are isolates of the Caulobacteriaceae including members of the genus Gallionella and Siderophacus.

While the foregoing is directed to embodiments, versions and examples of the present disclosure, which are included to enable a person of ordinary skill in the art to make and use the disclosures when the information in this patent is combined with available information and technology, the disclosure is not limited to only these particular embodiments, versions and examples. Also, it is within the scope of this disclosure that the aspects and embodiments disclosed herein are usable and combinable with every other embodiment and/or aspect disclosed herein, and consequently, this disclosure enabling for any and all combinations of the embodiments and/or aspects disclosed herein. Other and further embodiments, versions and examples of the disclosure may be devised without departing from the basic scope thereof and the scope thereof is determined by the claims that follow.

1. A method of controlling a deleterious bacteria in a fluid, comprising:
injecting a nitrite or nitrate into the fluid;
identifying a phage capable of infecting the deleterious bacteria; and
injecting the phage into the fluid.

2. The method of claim 1 wherein the nitrite or nitrate is selected from the group consisting of potassium nitrate, potassium nitrite, sodium nitrate, sodium nitrite, ammonium nitrate, and mixtures thereof.

3. The method of claim 1, wherein the deleterious bacteria is a sulfate reducing bacteria (SRB).

4. The method of claim 1, further comprising introducing a nitrate-reducing bacteria or nitrate reducing sulfide oxidizing bacteria (NRB) into the fluid.

5. The method of claim 4 wherein the NRB is selected from a library.

6. The method of claim 1 wherein the NRB is selected from the group consistent consisting of Campylobacter sp. Nitrobacter sp., Nitrosomonas sp., Thiomicrospira sp., Sulfurospirillum sp., Thauera sp., Paracoccus sp., Pseudomonas sp., Rhodobacter sp., Desulfovibrio sp., and mixtures thereof.

7. The method of claim 6 wherein NRB is selected from the group consisting of Nitrobacter vulgaris, Nitrosomonas europea, Pseudomonas stutzeri, Pseudomonas aeruginosa, Paracoccus denitrificans, Sulfurospirillum deleyianum, and Rhodobacter sphaeroides.

8. The method of claim 1 further comprising:
injecting 9,10-anthraquinone, a molybdate, or a molybdate salt into the fluid.

9. The method of claim 8 wherein the molybdate salt is sodium molybdate, lithium molybdate, or mixtures thereof.

10. The method of claim 1 further comprising prior to the step of injecting an inorganic nitrate into the fluid:
injecting a biocide into the fluid.

11. The method of claim 10, wherein the biocide and phage are mixed prior to injection.

12. The method of claim 10 wherein the biocide is bleach, chlorine dioxide, DBNPA, peracetic acid, a quaternary amine compound, THPS, dimethyloxazolidine, or an aldehyde-based biocides.

13. The method of claim 10 wherein the phage is isolated from an environment containing the deleterious bacteria.

14. The method of claim 10 wherein the phage is selected from phages known to be effective against the deleterious bacteria.

15. The method of claim 10 wherein phage is optimized by altering its host range determination, growth characteristics, or phage production.

16. The method of claim 1, wherein the nitrite or nitrate is injected in sufficient quantities to achieve a concentration of between 500 ppm and 1000 ppm by weight of fluid.

17. The method of claim 4, wherein NRB is injected in sufficient quantities to achieve a concentration of NRB between 10¹ and 10⁴ bacteria count/ml of the fluid.
